(19) United States
(12) Patent Application Publication
(10) Pub. No.: US 2022/0409290 A1
(43) Pub. Date: Dec. 29, 2022

(54) METHOD AND SYSTEM FOR REPRODUCING AN INSERTION POINT FOR A MEDICAL INSTRUMENT

(71) Applicant: ATLAS MEDICAL TECHNOLOGIES GMBH, Bochum (DE)

(72) Inventor: Timo KRÜGER, Berlin (DE)

(21) Appl. No.: 17/775,663

(22) PCT Filed: Nov. 11, 2020
(86) PCT No.: PCT/EP2020/081715

§ 371 Date: May 10, 2022

(30) Foreign Application Priority Data
Nov. 11, 2019 (DE) 10 2019 130 368.0

(51) Int. Cl. A61B 34/20; A61B 90/00

(52) U.S. Cl. CPC A61B 34/20; A61B 90/361; A61B 2034/2055

(57) ABSTRACT

The invention relates to a method for displaying an injection point for a medical instrument. The method comprises the following steps:
Providing at least one marker on a surface of an object, with such marker exhibiting the property that it can be recorded both tomographically, in particular fluoroscopically, and also optically;
Generating tomographic image data that can be used to reconstruct a fluoroscopic image of the at least one marker, located on the surface of the object, together with the object;
Determining the insertion point for the medical instrument on the surface of the object relative to the at least one marker in the coordinate system of the tomographic image data;
Generating visual image data that can be used to reconstruct a visual image of the at least one marker, located on the surface of the object, together with the object;
Transforming the coordinate of the insertion point in the coordinate system of the tomographic image data into the coordinate system of the visual image data using the relative position of the insertion point to the at least one marker; and
Displaying the insertion point for the medical instrument in real time in a view of the object.

The invention relates to a method for displaying an insertion point for a medical instrument. The invention further relates to a medical system for displaying an insertion point for a medical instrument.

Punctures are regularly carried out in diagnostics and also in therapy. A puncture is a targeted insertion of a medical instrument, in particular a needle, e.g. a hollow needle or a probe, into the human body. This means that the medical instrument is inserted into the human body and directed to a target location inside the human body, so that e.g. energy can be applied, liquid or tissue samples can be taken, or medication can be injected there.

A puncture is regularly carried out under visual control in particular if the target location comprises sensitive body tissues, e.g. nerve or organ tissue, or if sensitive body tissue is located near the target location. A puncture under visual control typically comprises that the position and orientation of the medical instrument inside the human body is recorded using imaging systems, such as computed tomography (CT), magnetic resonance imaging (MRI), or sonography.

A puncture under visual control can in particular be complemented with a positioning device that can be used to display, and especially mark, the insertion point and insertion angle of the medical instrument.

EP 1 887 960 B1, for example, describes a positioning device for positioning instruments within an examination area, wherein such positioning device can be used to mark, by means of targeted electromagnetic radiation, an access area and a relative orientation of an instrument in order to reach a target area located in the trajectory of the targeted electromagnetic radiation.

The underlying object of the invention is to provide an improved method for displaying an insertion point for a medical instrument. A further underlying object of the invention is to provide an improved system for displaying an insertion point for a medical instrument.

Regarding the method, the object is accomplished by means of a method for displaying an insertion point for a medical instrument, comprising the following steps:
Providing at least one marker on a surface of an object, with such marker exhibiting the property that it can be recorded both fluoroscopically and optically;
Generating fluoroscopic and/or tomographic image data that can be used to reconstruct a fluoroscopic and/or tomographic image of the at least one marker, located on the surface of the object, together with the object;
Determining the insertion point for the medical instrument on the surface of the object relative to the at least one marker in the coordinate system of the fluoroscopic and/or tomographic image data;
Generating visual image data that can be used to reconstruct a visual image of the at least one marker, located on the surface of the object, together with the object;
Transforming the coordinate of the insertion point in the coordinate system of the fluoroscopic and/or tomographic image data into the coordinate system of the visual image data using the relative position of the insertion point to the at least one marker;
Displaying the insertion point for the medical instrument in real time in a view of the object.

In the context of this description, a fluoroscopic and/or tomographic image and fluoroscopic and/or tomographic image data are to be understood as such images and thereby generated image data that are generated using imaging modalities, such as X-ray devices (e.g. C-arm), X-ray tomographs (computer tomographs), magnetic resonance tomographs, sonography devices, or the like. Images recorded using a computer tomograph are both fluoroscopic and tomographic images, while the term fluoroscopy is usually not used in connection with magnetic resonance imaging.

For the purposes of the invention described here, the term “tomographic image data” is also used for fluoroscopic image data, which is not tomographic image data in the narrower sense, but generated by an imaging modality like a C-arm, for example. Accordingly, in the following, the term tomographic images means all images generated by an imaging modality, i.e. also fluoroscopic images, for example from a C-arm.

“Displaying” refers to the displaying of at least the insertion point and, if known, also of the insertion angle and/or a puncture depth in a view of the object to be punctured. In the view of the object, the position of the insertion point is marked by its display on the surface of the object.

The view of the object can be a direct, real view of the object, and the display of the insertion point can be a marker projected onto the real surface, for example. Alternatively, the view of the object to be punctured can also be a real-time image display of the object on a monitor or on virtual reality glasses where the insertion point is displayed in real time. The view of the object can also be a real-time image display of the object on a transparent optical display where the insertion point is displayed perspectively correct in real time as augmented reality.

The medical instrument is in particular a cannulated medical instrument, for example a hollow needle. Alternatively, the medical instrument can be a needle-shaped probe that is used for interstitial thermotherapy, for example.

The insertion point is located on the real surface of the object to be punctured and defines in particular the position where the medical instrument is inserted into the object for a puncture. In addition, the insertion angle and/or puncture depth can be displayed in the view of the object in real time. The insertion angle indicates the angle, in relation to the surface, at which the medical instrument is inserted into the object for a puncture. The puncture depth indicates the distance to be covered by the medical instrument inserted into the insertion point at the insertion angle in order to reach a target area inside a human body.

The X-ray source and X-ray detector of an X-ray device can be used to generate tomographic image data. In order to reconstruct a tomographic image of the at least one marker located on the surface of the object together with the object from the tomographic image data, the object is positioned between the X-ray source and the X-ray detector so that X-rays emitted by the X-ray source penetrate the object and are then attenuated to different degrees depending on the inner structure of the object before they are detected by the X-ray detector. The tomographic image reconstructed from the tomographic image data can be a two-dimensional or a three-dimensional tomographic image.

The insertion point can be determined manually or automatically, e.g. software-based, in the tomographic image. The insertion point is preferably determined in such a way that the distance to be covered by the medical instrument inside the object in order to reach the target location is as short as possible. The insertion point is preferably determined in such a way that sensitive tissue is not damaged during a puncture.

The coordinate of the determined insertion point for the medical instrument on the surface of the object is preferably calculated mathematically by a computing unit in the coordinate system of the tomographic image data. Since the tomographic image reconstructed from the tomographic image data shows the object, and in particular the specified insertion point, together with the marker, the coordinate of the specified insertion point can be determined relative to the at least one marker. This means that the spatial relationship, i.e. the respective relative positions, between the insertion point and the marker is known in the coordinate system of the tomographic image data. Typically, a tomographic image does not show the complete object, but in particular the partial area of the object where the target location for the medical instrument is located. The marker is in particular provided in such a way, i.e. the marker is positioned in such a way, that it is visible together with the target location in a reconstructed tomographic image.

The visual image data can be generated by a camera. A still visual image of the surface together with the positioned marker or, as is preferred in the method according to the invention, moving visual images of the surface together with the positioned marker can be reconstructed from the generated visual image data.

Since the spatial relationship between the insertion point and the marker in the coordinate system of the tomographic image data can be determined and is then known, and the position and orientation of the marker in the coordinate system of the visual image data can be determined, it is possible, in particular when using the relative position of the insertion point to the at least one marker, to transform the coordinate of the insertion point in the coordinate system of the tomographic image data into the coordinate system of the visual image data. The coordinate of the insertion point and also the spatial relationship to the marker are then known in the coordinate system of the visual image data.

Transformation of the coordinate of the insertion point in the coordinate system of the tomographic image data into the coordinate system of the visual image data is possible in particular because the position of the marker both in the coordinate system of the tomographic image data and in the coordinate system of the visual image data is known and can thus be used as a reference for the transformation of coordinates from one coordinate system into the respective other coordinate system. The position of the insertion point in the coordinate system of the visual image data can in particular be used to display the insertion point in real time in the view of the object.

A user can reliably and precisely puncture the object based on the real-time display of the insertion point in the view of the object. A display in real time means in particular that a possible delay in the display cannot be resolved by the human eye, which means it would not be detected by the user. Preferably, the display of the insertion point in real time is adjusted to a changing view of the object, i.e. perspectively correct. The view of the object can be a real view or a reconstructed view. A real view can be an immediate, direct view of the real surface or an indirect view through a transparent medium, e.g. a transparent optical display. A reconstructed view can be a still visual image reconstructed from the visual image data. A reconstructed view of the object can also comprise a real-time image display reconstructed from the visual image data, i.e. moving visual images of the surface. Visual image data that can be used to reconstruct moving visual images can be generated by means of video technology, for example with a video camera. A video camera can be designed to generate three-dimensional visual image data from which three-dimensional moving visual images can be reconstructed.

The method according to the invention enables a precise display of the insertion point on the surface in a view of the object so that a user can puncture the object in a targeted and controlled manner. The advantage of the method is the fact that, during a puncture of the object, it is not necessary to take any X-ray images, or only a few X-ray images, of the object. It may actually suffice to only take one X-ray image prior to the puncture to specify the insertion point. In particular if the insertion angle and puncture depth are also displayed in the view of the object, it is generally not necessary to take an X-ray image after the puncture to check whether the medical instrument has actually reached the target location. Overall, depending on the particular application, it is possible to considerably reduce the radiation exposure for an object, in particular for a patient, using the method according to the invention.

A further advantage of the method according to the invention is the fact that, aside from an X-ray device that is already available anyway, no further bulky devices that would use additional space in an operating room are required. All that is needed to implement the method according to the invention is a marker, a camera, and a computing unit with the corresponding software. A doctor supported by the method according to the invention in puncturing an object in a reliable and precise manner is not hindered or restricted in his/her movements by additional bulky devices. An operating room does not have to be converted or modified, e.g. no devices have to be bolted to a wall or ceiling of the operating room in order to implement the method according to the invention.

The method according to the invention can also be implemented without a laser that is used to mark the insertion point with a laser beam. The advantage of displaying the insertion point in the view of the object without a laser is that a doctor does not need to pay attention to not block the laser beam which would result in the insertion point marked by the laser beam not being visible anymore.

Preferred embodiments of the method according to the invention for displaying an insertion point for a medical instrument are described in the following.

Preferably, the visual image data is generated as three-dimensional visual image data. Three-dimensional visual image data can, for example, be generated using a light field camera, a stereo camera, a triangulation system, or a time-of-flight (TOF) camera. The three-dimensional visual image data can be used to generate three-dimensional visual images that display, in addition to the insertion point, in particular also the insertion angle for the medical instrument in a perspectively correct manner.

In preferred embodiments of the method according to the invention, the method comprises the following steps:
Determining an insertion angle and/or a puncture depth for the medical instrument relative to the at least one marker in the coordinate system of the fluoroscopic and/or tomographic image data;
Transforming the insertion angle and/or the puncture depth determined in the coordinate system of the fluoroscopic and/or tomographic image data into the coordinate system of the visual image data using a relative orientation of the insertion angle and/or using a relative distance of the puncture depth to the at least one marker; and
Displaying the insertion angle and/or the puncture depth for the medical instrument in real time in the view of the object.

Preferably, the insertion point, insertion angle and puncture depth are displayed together in real time and perspectively correct in the view of the object. A doctor can then see at a glance where on the surface, at what angle, and up to what depth the object is to be punctured.

In the method according to the invention, it is preferred that the visual image data is generated continuously and that at least the insertion point determined in the coordinate system of the fluoroscopic and/or tomographic image data is transformed into the coordinate system of the respectively last generated visual image data. The display of at least the insertion point is preferably shown for the medical instrument in the view of the object in real-time.

Moving visual images can be reconstructed as a real-time image display from the continuously generated visual image data. Because the visual image data is generated continuously, a relative movement of the object can be recorded and the insertion point, insertion angle and/or puncture depth can be displayed in real time and perspectively correct in the view of the object.

In particular, the coordinate of the insertion point in the coordinate system of the tomographic image data as well as the insertion angle and/or the puncture depth can be transformed into the coordinate system of the last generated visual image data, and then displayed in real time.

The generated visual image data can be used to reconstruct a visual image of the surface that displays the insertion point for the medical instrument. A visual image can be a two- or three-dimensional still visual image or, which is preferred, a two- or three-dimensional visual image of moving visual images. The visual image can be displayed on a monitor. In addition, the visual image can display the insertion angle and/or the puncture depth for the medical instrument.

The insertion point for the medical instrument can also be displayed in an indirect view of the object on a transparent optical display. The view of the real surface is visible through the transparent optical display, wherein the insertion point is displayed on the transparent display perspectively correct in relation to the view of the real surface.

The optical display can be mounted on a frame similar to eyeglasses, which the user can put on so that the optical display is located in front of the eyes of the user. The user can then view the real object through the transparent optical display, i.e. the user can indirectly see the real image of the surface. The insertion point for the medical instrument can be displayed perspectively correct in relation to the view of the real surface on the transparent optical display in real time, so that a user sees the insertion point on the surface of the object in the indirect view of the object. In addition to the insertion angle, the insertion angle and/or the puncture depth for the medical instrument can be displayed on the transparent optical display. The frame with optical display preferably has a camera mounted on it that can continuously generate visual image data. The insertion point, the insertion angle and/or the puncture depth can then be displayed in real time in the indirect view of the object in such a way that the insertion point, the insertion angle and/or the puncture depth are displayed perspectively correct in relation to the view of the real surface.

In addition, or alternatively, the insertion point for the medical instrument can be displayed as an optical marker on the real surface of the object. For example, the insertion point can be displayed directly on the real surface of the object, in particular projected onto it, as an optical marker by means of a laser beam or by means of an alignment crosshair generated by a video projector The laser and/or video projector are then preferably autocalibrated by a camera used to generate visual image data. In addition, the insertion angle and/or the puncture depth can also be displayed as an optical marker.

In some embodiments, the insertion point and the insertion angle and/or the puncture depth are displayed in the form of a digital representation of a virtual tool in real time in the view of the object. In particular, the virtual tool can be displayed in real time and perspectively correct on a transparent optical display or on a monitor in a real-time image display of the object.

Especially in embodiments of the method according to the invention where the insertion point and the insertion angle and/or the puncture depth are displayed in the form of a digital representation of a virtual tool, the method can comprise the following steps:
Optical recording of position and orientation of the medical instrument relative to the at least one marker in the coordinate system of the generated visual image data;
Determining whether the recorded position and orientation of the medical instrument corresponds to the position and orientation of the displayed virtual tool, and if this is the case:
Signaling that the recorded position and orientation of the medical instrument corresponds to the position and orientation of the displayed virtual tool.

A method which includes signaling that the recorded position and orientation of the medical instrument corresponds to the position and orientation of the displayed virtual tool has the advantage of a user receiving feedback whether the medical instrument is oriented relative to the surface in such a way that the object can be punctured along the specified path.—The fact that the recorded position and orientation of the medical instrument corresponds to the position and orientation of the displayed virtual tool can, for example, be signaled optically, e.g. in the view of the object, or acoustically.

If the recorded position and orientation of the medical instrument does not correspond to the position and orientation of the displayed virtual tool, the method can comprise calculating a trajectory between the recorded position and orientation of the medical instrument and the position and orientation of the displayed virtual tool.

For example, the calculated trajectory can be used to display a virtual directional indication in the view of the object in real time. The directional indication preferably shows the direction in which the medical instrument has to be moved in order to achieve alignment of the position and orientation of the medical instrument with the position and orientation of the virtual tool displayed in the view of the object.

The virtual directional indication can support a user in aligning the position and orientation of the medical instrument with the position and orientation of the displayed virtual tool.

In embodiments of the method according to the invention where the insertion point and the insertion angle and/or the puncture depth can be displayed in the form of a digital representation of a virtual tool, the method preferably comprises the following step:
Aligning the digital representation of the displayed virtual tool in real time relative to the at least one marker in relation to a recording axis, along which the visual image data is generated.

Aligning of the digital representation of the displayed virtual tool in real time relative to the at least one marker in relation to a recording axis, along which the visual image data is generated, enables a perspectively correct display of the tool in real time in the view of the object.

A relative movement of the object or a relative change of perspective on the surface in the view of the object can be taken into consideration by aligning the digital representation of the displayed virtual tool in real time in relation to the recording axis, so that the insertion point, insertion angle and/or puncture depth are always displayed perspectively correct in the view. A user and the object can then move relative to one another, and the user can rely on the fact that the insertion point, insertion angle and/or puncture depth are correctly displayed in the view at all times.

With regard to the medical system, the task mentioned initially is solved by means of a medical system for displaying an insertion point for a medical instrument. The medical system features a marker, an imaging modality, in particular an X-ray device or a computer tomograph, a camera, a computing unit, and a display unit.

The marker is designed in such a way that it can be recorded both fluoroscopically and/or tomographically as well as optically. The imaging modality, in particular the X-ray device, is designed to generate fluoroscopic or/and tomographic image data and usually comprises an X-ray source and an X-ray detector. The X-ray device can be a computed tomograpy (CT) device or a C-arm device, for example. However, the imaging modality can also be a sonography device or a magnetic resonance tomograph, for example The camera is designed to generate visual image data; it can be a light field camera, a stereo camera, a triangulation system, or a TOF camera, for example. The camera is preferably designed in such a way that image data, in particular three-dimensional image data, can be generated continuously. A scanner can also be provided instead of a camera, and the visual image data can be generated using a light section process.

The computing unit is designed to
Determine the insertion point for the medical instrument on the surface of the object relative to the at least one marker in the coordinate system of the fluoroscopic and/or tomographic image data; and
Transform the coordinate of the insertion point in the coordinate system of the fluoroscopic and/or tomographic image data into the coordinate system of the visual image data using the relative position of the insertion point to the at least one marker.

The display unit is designed to display the insertion point for the medical instrument in real time in a real or reconstructed view of the object.

The medical system according to the invention is in particular designed in such a way that it can be used to implement the method according to the invention for displaying an insertion point for a medical instrument.

The camera and X-ray device are both operatively connected to the computing unit so that the computing unit can access, and then process, visual image data generated by the camera and tomographic image data generated by the X-ray device. Furthermore, the computing unit in particular is operatively connected to the display unit for visualizing the insertion point for the medical instrument in real time in the view of the object.

Preferred embodiments of the medical system according to the invention for displaying an insertion point for a medical instrument are described in the following.

The computing unit can be designed as an electronic data processing system or as a component of an electronic data processing system, and features in particular a CPU (Central Processing Unit), a memory, and a computer-readable storage medium with permanently stored computer programs.

The computing unit and/or the X-ray device can be designed to reconstruct a tomographic image from the tomographic image data. The computing unit and/or a separate data processing system can be designed to reconstruct a visual image from visual image data generated by the camera.

The display unit can be an optical display that is operatively connected to the computing unit and on which the insertion point for the medical instrument can be visualized by means of the computing unit. The optical display can, in particular, be part of an augmented reality system. For example, the optical display can be a component of glasses to which the camera is also attached. The optical display can be transparent so that the real surface can be viewed indirectly through the optical display and, at the same time, the insertion point can be displayed in the indirect view.

The display unit can be a monitor that is operatively connected to the computing unit, for example a computer monitor or a monitor of a virtual reality (VR) system, e.g. VR glasses. The monitor can display, in a perspectively correct manner, a reconstructed view of the object, for example as a real-time image display of the object together with at least the insertion point

The display unit can also be a video projector that is autocalibrated with the camera and designed to display the insertion point for the medical instrument as an optical marker on the real surface of the object. The video projector is preferably autocalibrated with the camera and designed to project an alignment crosshair on the real surface of the object, the center of which represents the position of the insertion point. An insertion point can also be displayed simultaneously in an indirect view of the object and, projected onto the real surface, as an optical marker. The redundancy may allow for the insertion point to be displayed with a comparatively higher degree of reliability.

The at least one marker can be bendable and flexible and created with e.g. adhesive tape that can be adhesively applied to the real surface of the object. In order to implement the method according to the invention, the adhesive tape can be used to adhesively apply a regular or irregular pattern on the surface, thus creating the marker.

The marker can also be created using double-sided adhesive foil or double-sided adhesive paper with a pre-cut pattern. The adhesive foil can be glued to the real surface, and the carrier film can then be removed, leaving only the pre-cut pattern on the surface creating the marker.

If the marker is provided on a carrier paper or a carrier foil, the marker itself can also consist of several not directly connected components, with their relative position to one another also being determined by the carrier foil or the carrier paper. If, in the application, the carrier foil or the carrier paper is removed from the marker after the marker has been applied to a body surface, the components of the marker will respectively maintain their relative position.

However, the marker can also be rigid and available e.g. in the form of a solid block that can be adhesively applied to the body surface in the application.

Preferably, the adhesive tape or the adhesive foil contains a metal, like titanium or stainless steel or, alternatively, a material like BaSOₓ, and in particular barium sulfate (BaSO₄), so that the adhesive tape or the adhesive foil can be detected tomographically, in particular fluoroscopically.

The at least one marker can also feature at least one tomographically and in particular fluoroscopically detectable element and/or at least one optically detectable element. The tomographically detectable element can be made up of a metal and designed in such a way that it can be identified as a tomographically or fluoroscopically detectable element in a tomographic or fluoroscopic image. For example, metal balls that can be identified in a fluoroscopic and/or tomographic image can be arranged across the surface of the marker. The optically detectable element can be a light emitting diode that is designed to emit electromagnetic radiation in a defined wavelength range. In particular, several light emitting diodes can be distributed across the area of the marker. The defined wavelength range preferably comprises infrared radiation. The camera then preferably features an infrared sensor for detecting the infrared radiation emitted by the light emitting diode. The tomographically detectable elements and the optically detectable elements preferably have a known spatial relationship to one another. It is also possible to use elements that can be detected both tomographically and optically. For example, metal balls can also be used as optically detectable elements.

The medical system can also comprise a robotic arm that is designed to hold the medical instrument and use it to carry out a puncture. Preferably, the robotic arm is designed to carry out a software-controlled puncture according to the specified insertion point, insertion angle, and puncture depth. The camera can be used to optically detect the position and orientation of the robotic arm during the puncture, and the computing unit can then evaluate them.

The invention also relates to a computer program that is designed to determine an insertion point for a medical instrument on a surface of an object relative to a marker in the coordinate system of generated tomographic image data and to transform the coordinate of the insertion point in the coordinate system of the tomographic image data into the coordinate system of generated visual image data using a relative position of the insertion point to the marker. By executing the computer program, it is possible to implement in particular the steps of “determining the insertion point for the medical instrument” and “transforming the coordinate of the insertion point” of the method according to the invention.

The invention further relates to a computer-readable storage medium where the computer program according to the invention is permanently stored. The computer-readable storage medium is preferably an element of the computing unit, and the stored computer program can preferably be loaded into a memory and processed and executed by processors.

The invention will now be explained in more detail using schematically depicted exemplary embodiments and referencing the figures. The figures show the following:

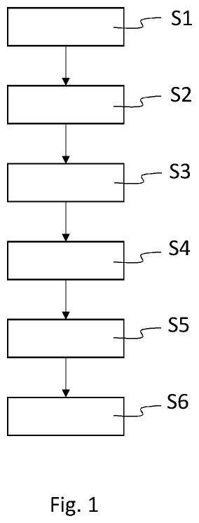

FIG. 1: A flow chart of a method for displaying an insertion point for a medical instrument.

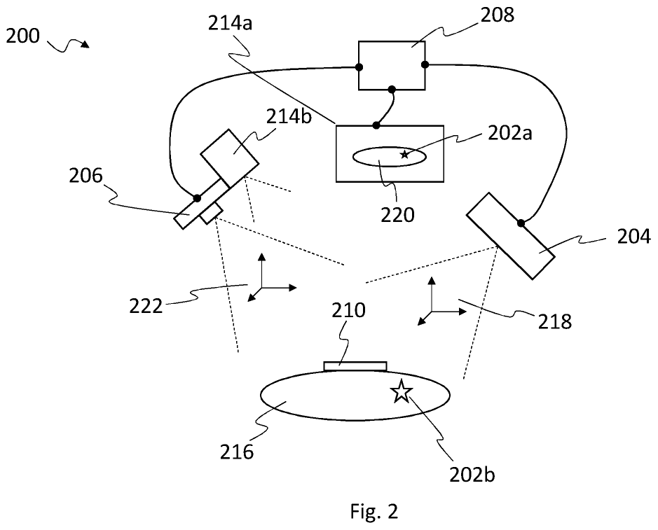

FIG. 2: A schematic diagram of a medical system for displaying an insertion point for a medical instrument.

FIG. 1 shows a flow chart of a method for displaying an insertion point for a medical instrument.

The sequence of the method is as follows:

Initially (step S1), at least one marker is provided on a surface of an object. The properties of the marker allow for tomographic, in particular fluoroscopic, and also optical detection. For example, the marker can be created with adhesive tape that is adhesively attached to the surface in a regular or irregular pattern, thereby creating the marker. In order for the marker to be tomographically detectable, it will preferably have barium sulfate, which is visible in a fluoroscopic image of the marker, distributed across the surface or in selected areas of the adhesive tape. The marker can also be provided on the surface of the object by adhesively attaching double-sided adhesive foil with a precut pattern to the surface. The carrier foil of the double-sided adhesive foil can be peeled off in such a way that only adhesive tape remains in the pre-cut pattern on the surface, thereby creating the marker. The marker is thus created by a predefined pattern of the adhesive foil. For example, a detected deformation of the predefined pattern can indicate a movement of the object. The marker can also be created with a carrier material that has fluoroscopically and optically detectable elements arranged on it. The fluoroscopically detectable elements can be metal balls, and the optically detectable elements can be light emitting diodes, for example. The fluoroscopically and optically detectable elements are preferably arranged in a known spatial relationship to one another.

Subsequently (step S2), tomographic image data is generated that can be used to reconstruct a fluoroscopic image of the at least one marker, arranged on the surface of the object, together with the object. The tomographic image data can be generated with an X-ray device, for example, which features an X-ray source and an X-ray detector. To generate the tomographic image data, the object is arranged between the X-ray source and the X-ray detector in such a way that the X-rays emitted by the X-ray source penetrate the marker and at least that partial area of the object where a target area to be punctured is located, and are then detected by the X-ray detector.

Subsequently (step S3), the insertion point for the medical instrument on the surface of the object is determined relative to the at least one marker provided on the surface in the coordinate system of the tomographic image data. For example, the insertion point for the medical instrument can initially be determined in a fluoroscopic image reconstructed from the tomographic image data, e.g. implemented by a doctor or using software. A computing unit can then mathematically determine the coordinate of the specified insertion point in the coordinate system of the tomographic image data. Since the position of the marker in the coordinate system of the tomographic image data is known, the spatial relationship between the marker and the insertion point in the coordinate system of the tomographic image data can be determined. In particular, the relative position of the insertion point to the marker in the coordinate system of the tomographic image data is then known.

In addition to the insertion point, the insertion angle and/or puncture depth for the medical instrument relative to the at least one marker in the coordinate system of the tomographic image data can also be determined. It is then known in the coordinate system of the tomographic image data where, at what angle and how deep the medical instrument should be inserted into the object for a puncture.

Subsequently (step S4), visual image data is generated that can be used to reconstruct a visual image of the at least one marker, arranged on the surface of the object, together with the object. The visual image data is generated using a camera that is preferably designed to continuously create visual image data as three-dimensional visual image data.

Subsequently (step S5), the coordinate of the insertion point in the coordinate system of the tomographic image data is transformed into the coordinate system of the visual image data using the relative position to the at least one marker. If the insertion angle and/or the puncture depth were also determined in the coordinate system of the tomographic image data, transformation of the insertion angle, using the relative orientation of the insertion angle to the at least one marker, and/or of the puncture depth, using the relative distance of the puncture depth to the at least one marker, into the coordinate system of the visual image data is also performed.

Subsequently (step S6), the insertion point for the medical instrument and—if identified—also the insertion angle and/or the puncture depth are displayed in a view of the object. If available, the insertion point, insertion angle and puncture depth for the medical instrument are preferably displayed together in the view of the object.

The view of the object can be a real view or a reconstructed view. A real view can be a direct view of the real surface or an indirect view of the real surface, for example through a transparent optical display. In a direct view of the object, the insertion point for the medical instrument can be displayed by means of an optical marker, for example. In an indirect view of the object through a transparent optical display, the insertion point for the medical instrument can be displayed in such a way that it is displayed in real time and perspectively correct in relation to the surface. Furthermore, in the indirect view of the object, the insertion angle and puncture depth can also be displayed in real time and perspectively correct in relation to the surface. It is possible to display the insertion point, insertion angle and puncture depth in the form of a digital representation of a virtual tool in real time in the indirect view of the object. A reconstructed view of the object can be a photograph reconstructed from generated visual image data, in particular a real-time image recording of the object. The reconstructed view of the object can, for example, be displayed on a monitor, e.g. a computer monitor or the monitor of VR glasses. The reconstructed view can display the insertion point, insertion angle and puncture depth, e.g. in the form of a digital representation of a virtual tool.

It is possible to only display the insertion point in a single view. It is also possible to display the insertion point, insertion angle and puncture depth together in one view. It is also possible to display the insertion point, insertion angle and puncture depth in one view and, in an additional view, only the insertion point. In this case, the insertion point is displayed in two different views, i.e. redundantly. For example, it is possible to display the insertion point, insertion angle and puncture depth in the form of a digital representation of a virtual tool in an indirect view of the object and, in addition, the insertion point by means of an optical marker in a direct view. A user can then choose between the two views, for example. An optical marker can also be integrated into an indirect view of the object.

FIG. 2 shows a schematic diagram of a medical system 200 for displaying an insertion point for a medical instrument (not shown).

The medical system 200 comprises an X-ray device 204, a camera 206, a computing unit 208, a marker 210, and two display units 214a, 214b. The medical system 200 is in particular suitable for implementing the method described with reference to FIG. 1.

The marker 210 can be arranged on an object to be punctured 216 (not part of the medical system 200), for example a patient. The marker 210 is then preferably arranged on the object 116 in such a way that it follows a movement of the object, so that there is no relative movement between the object 216 and the marker 210. Preferably, the marker 210 is adhesively attached to the object 216. For example, the marker 210 can be created with adhesive tape that is adhesively attached to the surface of the object 216 in a regular or irregular pattern. The marker 210 is designed in such a way that it can be recorded both tomographically, in particular fluoroscopically, and also optically. In order for the marker 210 to be recorded optically, it is preferably created in a color and/or form that ensures a visible contrast to the surface of the object 210 in a visual image recording. In order for the marker 210 to be also visible in a tomographic recording, it can have barium sulfate as a contrast medium in defined areas.

The X-ray device 204 can be a computer tomograph (CT) device, for example, and comprises an X-ray source and an X-ray detector (not shown). In order to generate tomographic image data, the object 216 is arranged between the X-ray source and the X-ray detector of the X-ray device 204 in such a way that the generated tomographic image data can be used to reconstruct a tomographic image of the marker 210 together with the object 216. A reconstructed tomographic image can initially be used to plan a puncture of the object 216, for example to specify an insertion point on the surface of the object 210.

The computing unit 208 can determine the coordinate of the insertion point in the coordinate system of the tomographic image data 218 relative to the position of the marker 210. The computing unit 208 is also designed to determine the insertion angle and the puncture depth for the medical instrument in the coordinate system of the tomographic image data 218.

To determine the insertion point, the insertion angle and the puncture depth in the coordinate system of the tomographic image data 218, the computing unit accesses and processes the tomographic image data generated by the X-ray device 204. The computing unit 208 is also operatively connected to the camera 206 to access and process visual image data generated by the camera.

So that the insertion point 202a, the insertion angle and the puncture depth can be displayed in a view 220 of the surface of the object 216, the computing unit 208 is designed to transform the coordinate of the insertion point determined in the coordinate system of the tomographic image data, as well as the insertion angle and the puncture depth into the coordinate system 222 of the visual image data generated by the camera 206. The computing unit 208 is designed to use the relative position of the insertion point to the at least one marker for transforming the coordinate of the insertion point. The relative position of the insertion point to the at least one marker 210 can be used for transforming the coordinate of the insertion point, because the position of the marker 210 is known in both the coordinate system of the tomographic image data 218 and in the coordinate system of the visual image data 222. The position of the marker 210 can thus be used as a reference for transforming the coordinate of the insertion point from the coordinate system of the tomographic image data 218 into the coordinate system of the visual image data 222.

The computing unit 208 is further operatively connected to the display unit 214a and designed to display the insertion point 202a for the medical instrument in real time and perspectively correct in a view 220 of the surface.

The medical system 200 features two display units 214a, 214b. The medical system 200 can also have only one of the two display units 214a, 214b, or an alternative display unit. The display unit 214b is a video projector that is autocalibrated with the camera 206 and designed to display the insertion point 202b as an optical marker.

The display unit 214a is a transparent optical display that can be mounted, together with the camera 206, on a frame, e.g. an eyeglasses frame.

The view 220 on the transparent optical display 214a is an indirect view of the real surface of the object 216 that shows the insertion point 202a. The insertion point 202a can be displayed in real time and perspectively correct in the view 220. Instead of the optical display 214a, or in addition to it, the medical system 200 can also feature a monitor that displays the insertion point in a reconstructed view of the object.

What is claimed is:

1. A method for displaying an insertion point for a medical instrument, with such method comprising the following steps:
Providing at least one marker on a surface of an object, with such marker exhibiting the property that it can be recorded both tomographically, in particular fluoroscopically, and optically;
Generating fluoroscopic or/and tomographic image data that can be used to reconstruct a fluoroscopic or/and tomographic image of the at least one marker, located on the surface of the object, together with the object;
Determining the insertion point for the medical instrument on the surface of the object relative to the at least one marker in the coordinate system of the fluoroscopic or/and tomographic image data;
Generating visual image data that can be used to reconstruct a visual image of the at least one marker, located on the surface of the object, together with the object;
Transforming the coordinate of the insertion point in the coordinate system of the fluoroscopic or/and tomographic image data into the coordinate system of the visual image data using the relative position of the insertion point to the at least one marker; and
Displaying the insertion point for the medical instrument in real time in a view of the object.

2. The method according to claim 1, wherein the visual image data is generated as three-dimensional visual image data

3. The method according to claim 1, comprising the following steps:
Determining an insertion angle and/or a puncture depth for the medical instrument relative to the at least one marker in the coordinate system of the fluoroscopic and/or tomographic image data;
Transforming the insertion angle or/and the puncture depth determined in the coordinate system of the fluoroscopic and/or tomographic image data into the coordinate system of the visual image data using a relative orientation of the insertion angle and/or using a relative distance of the puncture depth to the at least one marker; and
Displaying the insertion angle and/or the puncture depth for the medical instrument in real time in the view of the object.

4. The method according to claim 1, wherein the visual image data is generated continuously and at least the insertion point determined in the coordinate system of the fluoroscopic or/and tomographic image data is transformed into the coordinate system of the respectively last generated visual image data, and the display of at least the insertion point for the medical instrument in the view of the object is shown in real time.

5. The method according to claim 1, wherein the view of the object is a visual image of the surface that has been reconstructed from the visual image data generated, and the insertion point for the medical instrument is displayed in the visual image.

6. The method according to claim 1, wherein the insertion point for the medical instrument is displayed on a transparent optical display through which the view of the real surface is visible, wherein the insertion point is displayed on the transparent display perspectively correct in relation to the view of the real surface.

7. The method according to claim 1, wherein the insertion point for the medical instrument is displayed as an optical marker on the real surface of the object.

8. The method according to claim 3, wherein the insertion point and the insertion angle and/or the puncture depth are displayed in the form of a digital representation of a virtual tool in real time in the view of the object.

9. The method according to claim 8, comprising the following steps:
Optical recording of position and orientation of the medical instrument relative to the at least one marker in the coordinate system of the visual image data generated,
Determining whether the recorded position and orientation of the medical instrument corresponds to the position and orientation of the displayed virtual tool, and if this is the case:
Signaling that the recorded position and orientation of the medical instrument corresponds to the position and orientation of the displayed virtual tool.

10. The method according to claim 9, comprising the following steps:
If the recorded position and orientation of the medical instrument does not correspond to the position and orientation of the displayed virtual tool:
Calculating a trajectory between the recorded position and orientation of the medical instrument and the position and orientation of the displayed virtual tool; and
Displaying a virtual directional indication in real time in the view of the object, wherein the directional indication preferably shows the direction in which the medical instrument has to be moved in order to achieve alignment of the position and orientation of the medical instrument with the position and orientation of the virtual tool displayed in the view of the object.

11. The method according to claim 8, comprising the following steps:
Aligning the digital representation of the virtual tool in real time relative to the at least one marker in relation to a recording axis, along which the visual image data is generated.

12. A medical system for displaying an insertion point for a medical instrument, with such system comprising the following:
A marker that is designed in such a way that the marker can be recorded both tomographically, in particular fluoroscopically, and also optically;
An imaging modality for generating fluoroscopic or/and tomographic image data;
A camera for generating visual image data;
A computing unit that is designed to
Determine the insertion point for the medical instrument on the surface of the object relative to the at least one marker in the coordinate system of the fluoroscopic or/and tomographic image data;
Transform the coordinate of the insertion point in the coordinate system of the fluoroscopic or/and tomographic image data into the coordinate system of the visual image data using the relative position of the insertion point to the at least one marker; and
A display unit for displaying the insertion point for the medical instrument in real time in a real or reconstructed view of the object.

13. The medical system according to claim 12, wherein the camera is a light field camera, a stereo camera, a triangulation system, or a TOF camera.

14. The medical system according to claim 12, wherein the display unit is an optical display that is operatively connected to the computing unit and on which the insertion point for the medical instrument can be visualized by means of the computing unit.

15. The medical system according to claim 12, wherein the display unit is a video projector that is autocalibrated with the camera and designed to display the insertion point for the medical instrument on the real surface of the object as an optical marker.

16. The medical system according to claim 12, wherein the at least one marker is created with adhesive tape that can be adhesively attached on the surface of the object.

17. The medical system according to claim 16, wherein the adhesive tape contains BaSOₓ so that the adhesive tape can be detected fluoroscopically.

18. The medical system according to claim 12, wherein the at least one marker contains at least one fluoroscopically detectable element and/or at least one optically detectable element.

19. The medical system according to claim 18, wherein the fluoroscopically detectable element can be made up of a metal and is designed in such a way that it can be identified as a tomographically detectable element in a tomographic image.

20. The medical system according to claim 18, wherein the optically detectable element can be a light emitting diode that is designed to emit electromagnetic radiation in a defined wavelength range.

21. The medical system according to claim 20, wherein the defined wavelength range comprises infrared radiation and the camera features an infrared sensor for detecting the infrared radiation emitted by the light emitting diode.

22. A computer program that is designed to determine an insertion point for a medical instrument on a surface of an object relative to a marker in the coordinate system of generated tomographic image data and to transform the coordinate of the insertion point in the coordinate system of the fluoroscopic or/and tomographic image data into the coordinate system of generated visual image data using a relative position of the insertion point to the marker.

23. A computer-readable storage medium where the computer program according to claim 22 is permanently stored.
